(19) United States
(12) Patent Application Publication
(10) Pub. No.: US 2012/0093904 A1
(43) Pub. Date: Apr. 19, 2012

(54) COMPOSITION COMPRISING AN ANIONIC POLYMERIC MATERIAL AND THE SALT OF A SATURATED MONOCARBOXYLIC ACID HAVING 6 TO 22 CARBON ATOMS

(75) Inventors: Erna Roth, Darmstadt (DE); Ruediger Alexowsky, Nauheim (DE); Hans-Ulrich Petereit, Darmstadt (DE)

(73) Assignee: EVONIK ROEHM GMBH, Darmstadt (DE)

(21) Appl. No.: 13/378,112

(22) PCT Filed: Jul. 30, 2009
(86) PCT No.: PCT/EP09/59860

§ 371 Date: Dec. 14, 2011

(51) Int. Cl. A61K 8/02; A61K 8/81; A61K 9/00; A61K 47/32

(52) U.S. Cl. 424/401; 514/772.6; 424/400

(57) ABSTRACT

The invention relates to a coating composition for the coating or binding of pharmaceutically, nutraceutically or cosmetically active ingredients, comprising (a) an anionic polymeric material, and (b) one or more salts of saturated monocarboxylic acids having 6 to 22 carbon atoms, characterized in that the amount of the salts of the monocarboxylic acids in the composition corresponds to 3 to 50 mol percent of the amount of anionic groups in the polymeric material. The invention also relates to a process for preparing a dispersion out of the composition, as well as the use of said composition in enteric-coated solid dosage forms.

### FIELD OF THE INVENTION

The present invention refers to a coating or binding composition comprising an anionic polymeric material, particularly an anionic (meth)acrylate copolymer for faster and easier dispersion and as ready-to-use formulation, a gastric resistant, enteric-coated solid dosage form, and a process for preparing the same, as well as the use of said composition or aqueous dispersion for preparing the coating of gastric resistant, enteric-coated solid dosage forms.

### BACKGROUND OF THE INVENTION

Enteric coated products are designed to remain intact in the stomach and then to release the active substance in the upper intestine. Enteric coating can be applied to solid dosage forms, such as granules, pellets, capsules, or tablets. The purpose of enteric coating is to protect the stomach from irritating active compounds such as aspirin, or to improve drug bioavailability by preventing degradation of acid or gastric enzyme labile drugs.

### DESCRIPTION OF THE PRIOR ART

Several aqueous enteric-film coating systems are known. The document U.S. Pat. No. 6,420,473 refers to a non-toxic, edible, enteric film coating, dry powder composition for use in making an aqueous enteric suspension which may be used in coating pharmaceutical tablets, comprising an acrylic resin, an alkalizing agent capable of reacting with the acrylic resin such that, after reaction, 0.1 to 10 mole percent of the acidic groups are present in the salt form, and a detackifier. By this a fully-formulated, enteric film coating composition that may be readily dispersed in water and applied to pharmaceutical tablets was provided.

EP 1 101 490 B1 describes a pharmaceutical composition capable of releasing a drug at a target site in the intestine. The pharmaceutical composition comprises a core with a medical substance coated with a mixed film comprising a hydrophobic organic compound and an enteric polymer. The hydrophobic organic compound is preferably a higher fatty acid having 6 to 22 carbon atoms, which may have an unsaturated bond. It is explicitly stated that the hydrophobic organic compound is not a salt.

### PROBLEM AND SOLUTION

There is a desire for fully-formulated, enteric film coating compositions which are stable as aqueous dispersions and ready-to-use. Further, the aim was to provide compositions with improved dispersion time and which can be readily applied to pharmaceutical tablets.

The problem was solved by a

Composition for coating or binding of pharmaceutically, nutraceutically or cosmetically active ingredients, comprising (a) an anionic polymeric material, and (b) one or more salts of saturated monocarboxylic acids having 6 to 22 carbon atoms, characterized in that the amount of the salts of the monocarboxylic acids in the composition corresponds to 3-50 mol percent of the amount of anionic groups in the polymeric material.

The inventive composition may further comprise pharmaceutically, nutraceutically or cosmetically acceptable additives selected from the group consisting of antioxidants, brighteners, flavouring agents, flow aids, fragrances, glidants, penetration-promoting agents, pigments, plasticizers, polymers, pore-forming agents or stabilizers. Pharmaceutically, nutraceutically or cosmetically acceptable additives are well known to a person skilled in the art.

The pharmaceutically or nutraceutically composition according to the present invention preferably may be used as a coating agent for gastric resistant, enteric-coated pharmaceutical or nutraceutical solid dosage forms. The coating agent is a non-toxic, edible, enteric film coating and is having the form of either a dry powder composition or aqueous dispersion. In case of a dry powder composition it is for use in making an aqueous enteric suspension which may be used in coating pharmaceutical tablets, mini tablets, granules and cristalls.

In a most preferred embodiment the present invention provides a coating composition for the coating of cores comprising pharmaceutically, nutraceutically or cosmetically active ingredients in the form of a fully pre-formulated, enteric film coating composition for preparing a stable and ready to use aqueous dispersion which can be sprayed as a coating layer onto a core comprising a pharmaceutically, nutraceutically or cosmetically active ingredient to form a in a gastric resistant, enteric coated pharmaceutical nutraceutical or cosmetical drug form.

In another embodiment the present invention provides a fully pre-formulated binding composition for the binding of pharmaceutically, nutraceutically or cosmetically active ingredients in the form of a matrix formulation. The binding composition may be sprayed, for instance in a in a powder layering or granulation process, as a binding agent together with a pharmaceutically, nutraceutically or cosmetically active ingredient to form a in a matrix drug, for instance in the form of pellets, for pharmaceutical, nutraceutical or cosmetical purposes. In the form of a dry powder the coating and binding composition shows a reduced dispersion time and can be readily dispersed and then as a dispersion applied to pharmaceutical or nutraceutical solid dosage forms.

In a preferred embodiment compound (a) is an anionic (meth)acrylate copolymer consisting of free-radical polymerized units of C₁- to C₄-alkyl esters of acrylic or of methacrylic acid and (meth)acrylate monomers having an anionic group. Preferably, compound (a) is an anionic (meth)acrylate copolymer consisting of free-radical polymerized units of 25 to 95% by weight C₁- to Cₐ-alkyl esters of acrylic or of methacrylic acid and 5 to 75% by weight (meth)acrylate monomers having an anionic group. More preferred compound (a) is an anionic (meth)acrylate copolymer consisting of free-radical polymerized units of 45 to 75% by weight C₁- to C₄-alkyl esters of acrylic or of methacrylic acid and 25 to 55% by weight (meth)acrylate monomers having an anionic group.

The anionic polymer of compound (a) may be additionally partially neutralized by an alkaline agent, which is not a salt of the saturated monocarboxylic acids having 6 to 22 carbon atoms is selected from the group consisting of alkali metal salt and ammonium salt.

### Pharmaceutically, Nutraceutically or Cosmetically Active Ingredients

The inventive composition may be used for coating or binding of pharmaceutically, nutraceutically or cosmetically active ingredients. Pharmaceutically, nutraceutically or cosmetically active ingredients have in common that they are active ingredients which have a positive effect on the health of an organism, e.g the human health. They have also in common that their formulations are often the same or very similar. Often also the same kind of excipients or additives are used in combination with these kind of active ingredients. Pharmaceutically active ingredients are used to cure diseases and effect the health of an organism, e.g the human health more or less directly. Nutraceutical active ingredients are used to supplement the nutrition and thus support the health of an organism, e.g the human or animal health indirectly. Cosmetically active ingredients are meant to support the human health indirectly for instance by balancing the water content of the human skin.

### Salts of the Saturated Monocarboxylic Acids Having 6 to 22 Carbon Atoms

In a further preferred embodiment of the present invention the salt in respect to component (b) is selected from the group consisting of alkali metal salt or an ammonium salt. Preferably the salt of the saturated monocarboxylic acids having 6 to 22 carbon atoms is a water soluble salt or a water dispersible salt.

In a particularly preferred embodiment of the present invention, the salt in respect to component (b) is a salt of a saturated, preferably unbranched, preferably unsubstituted, mono carboxylic acid (fatty acid) having 6 to 22, preferably 6 to 10 or 16 to 20 carbon atoms, which may be selected from the group of consisting of the salts of caproic acid, ornathic acid, caprylic acid, pelargonic acid, caprinic acid, lauric acid, myristic acid, palmitic acid, margaric acid, stearic acid, arachidic acid or behenic acid or mixtures thereof. Even more preferred is an alkali metal salt or ammonium salt thereof. Even further preferred is a salt of caprylic acid, particularly preferred sodium caprylate or sodium stearate.

The salts of the following saturated monocarbonic acids are suitable for the purposes of the invention:

C₆: caproic acid (C₅H₁₁COOH),

C₇: oenanthic acid (C₆H₁₃COOH),

C₈: caprylic acid (C₇H₁₅COOH),

C₉: pelargonic acid (C₈H₁₇COOH),

C₁₀: capric acid (C₉H₁₉COOH),

C₁₂: lauric acid (C₁₁H₂₃COOH),

C₁₄: myristic acid (C₁₃H₂₇COOH),

C₁₆: palmitic acid (C₁₆H₃₁COOH),

C₁₇: margaric acid (C₁₆H₃₃COOH)

C₁₈: stearic acid (C₁₇H₃₅COOH),

C₂₀: arachidic acid (C₁₉C₃₉COOH),

C₂₂: behenic acid (C₂₁H₄₃COOH)

Salts of organic or anorganic acids other than salts of saturated, mono carboxylic acids (fatty acids) having 6 to 22 carbon atoms are assumed to be not suitable for the purposes the present invention.

Saturated, mono carboxylic acids (fatty acids) having 6 to 22 carbon atoms are not suitable for the purposes of the invention as long as they are not applied together with an alkali metal or an ammonium hydroxide to react in situ to the salt form (see examples 11 and 12).

The salt of a saturated, preferably unbranched, mono carboxylic acid (fatty acid) having 6 to 22, preferably 6 to 10 or 16 to 20 carbon atoms is preferably unsubstituted. Less preferred the salt of a saturated mono carboxylic acid (fatty acid) having 6 to 22, preferably 6 to 10 or 16 to 20 carbon atoms can be substituted with one hydroxyl group. In this exceptional case for instance sodium 2-hydroxy-octanoate (Na-2-hydroxy-octanoate) may be a suitable salt (see example 34).

It is understood that all the salts of a saturated, preferably unbranched, preferably unsubstituted, mono carboxylic acid (fatty acid) having 6 to 22, preferably 6 to 10 or 16 to 20 carbon atoms which are suitable in the sense of the present invention should be acceptable as a pharmaceutical ingredient.

### Amount of the Salts of Saturated Monocarboxylic Acids Having 6 to 22 Carbon Atoms

The amount of the salts of the monocarboxylic acids (fatty acids) in the composition or in the dispersion corresponds to 3-50 mol percent, most preferred 5-25 mol percent, even more preferred 5-15 mol percent, of the amount of the anionic groups present in the polymeric material. This should correspond to a degree of partial neutralization of 3-50 percent, most preferred 5-25 percent or even more preferred 5-15 percent, of the total amount of monomers with anionic groups present in the polymeric material, when (a) and (b) are brought together in a water containing environment. The certain amounts in percent by weight may be determined by using the known molecular weights of the polymeric material and the salts of the monocarboxylic acids components to calculate the mol percent ratios and the corresponding weight percent ratios. The suitable mol percent ratios and the corresponding weight percent ratios of the salts of monocarboxylic acids may be also be derived from the known acid value of the polymeric material.

The present invention also provides a process for preparing an aqueous coating dispersion, which dispersion is comprising an anionic polymeric material, in which the anionic groups are neutralized to a degree of 3 to 50 mol percent by one or more salt of saturated monocarboxylic acids having 6 to 22, preferably 6 to 10 or 16 to 20 carbon atoms. Said process is comprising the step of combining the anionic polymer and the salt of saturated monocarboxylic acids having 6 to 22 carbon atoms and water, mixing (homogenisation, for instance by vigorously stirring or by high pressure homogenisation) and obtaining the aqueous coating dispersion.

Pharmaceutically acceptable additives selected from the group consisting of pigments, release agents, plasticizers or emulsifiers may be are added to the dispersion. The Pharmaceutically acceptable additives may be added to the components (a) and (b) in the dry stage or to the already dispersed components (a) and (b) in the aqueous dispersion. The optionally pharmaceutically acceptable additives selected from the group consisting of pigments, release agents, plasticizers and emulsifiers may be involved in a way known to the skilled person, however without contributing to the invention per se.

### Gastric Resistant, Enteric-Coated Solid Dosage Form

The inventive composition may be used in the form of an aqueous dispersion to be sprayed as a coating layer onto a core comprising a pharmaceutically or nutraceutically active ingredient to a create a gastric resistant, enteric coated pharmaceutically or nutraceutically drug form.

Thus the invention discloses a gastric resistant, enteric coated pharmaceutically or nutraceutically drug form comprising a core with a pharmaceutically or nutraceutically active ingredient and a coating layer comprising a composition according to the invention.

The invention also discloses the use of the inventive composition for preparing the coating of gastric resistant, enteric-coated pharmaceutical or nutraceutical solid dosage forms.

### Anionic Polymeric Compounds

Suitable anionic polymeric materials may be cellulose acetate phthalate (CAP), cellulose acetate succinate (CAS), cellulose acetate trimelliate (CAT), hydroxypropyl methyl cellulose phthalate (HPMCP, HP50, HP55), hydroxypropylmethyl cellulose acetate succinate (HPMCAS-LF, -MF, -HF) or vinyl copolymers comprising structural units that are derived from unsaturated carboxylic acids other than acrylic acid or methacrylic acid as exemplified by polyvinylacetat phthalate or a copolymer of vinylacetate and crotonic acid 9:1. Polyacrylic acid, especially high molecular weight polyacrylic acid, especially crosslinked and/or noncrosslinked polyacrylic acid, is preferably not present in the inventive composition, because of its extremely high viscosity.

According to a preferred embodiment of the present invention the polymeric compound (a) is preferably selected from carboxyl functional (meth)acrylic polymers.

In a preferred embodiment compound (a) is an anionic (meth)acrylate copolymer consisting of free-radical polymerized units of C₁- to C₄-alkyl esters of acrylic or of methacrylic acid and (meth)acrylate monomers having an anionic group. Preferably, compound (a) is an anionic (meth)acrylate copolymer consisting of free-radical polymerized units of 25 to 95%, preferably 40 to 75 or 45 to 60 by weight C₁- to C₄-alkyl esters of acrylic or of methacrylic acid and 5 to 75, preferably 25 to 60 or 40 to 55% by weight (meth)acrylate monomers having an anionic group.

In a particularly preferred embodiment of the present invention, the salt in respect to component (b) is a salts of saturated mono carboxylic acids having 6 to 22 carbon atoms selected from the group consisting of caproic acid, ornathic acid, caprylic acid, pelargonic acid, caprinic acid, lauric acid, myristic acid, palmitic acid, margaric acid, stearic acid, arachidic acid or behenic acid or mixtures thereof, even more preferred an alkali metal salt thereof, even further preferred a salt of caprylic acid, particularly preferred sodium caprylate. Also preferred is sodium stearate.

The present invention provides an enteric-coated solid dosage form. As enteric-coated solid dosage form the dosage form according to the present invention is gastric resistant and shows less than 10 percent drug release in a simulated gastric fluid for at least 120 min according to USP 28. For example, this test for showing gastric resistance may be performed in a hydrochloric acid solution 0.1N, pH 1.2.

### Anionic (Meth)Acrylate Copolymer

In a preferred embodiment anionic (meth)acrylate copolymers are used for the coating. The anionic (meth)acrylate copolymer comprises 25 to 95, preferably 40 to 95, in particular 60 to 40, % by weight free-radical polymerized C₁- to C₄-alkyl esters of acrylic or of methacrylic acid and 75 to 5, preferably 60 to 5, in particular 40 to 60, % by weight (meth)acrylate monomers having an anionic group.

The proportions mentioned normally add up to 100% by weight. However it is also possible in addition, without this leading to an impairment or alteration of the essential properties, for small amounts in the region of 0 to 10, for example 1 to 5, % by weight of further monomers capable of vinylic copolymerization, such as, for example, hydroxyethyl methacrylate or hydroxyethyl acrylate, to be present. It is preferred that no further monomers capable of vinylic copolymerization are present.

C₁- to C₄-alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group is, for example, acrylic acid, with preference for methacrylic acid.

Suitable anionic (meth)acrylate copolymers are those composed of 40 to 60% by weight methacrylic acid and 60 to 40% by weight methyl methacrylate or 60 to 40% by weight ethyl acrylate (EUDRAGIT® L100 or EUDRAGIT® L 100-55 types).

EUDRAGIT® L100 is a copolymer of 50% by weight methyl meth-acrylate and 50% by weight methacrylic acid. The pH of the start of the specific active ingredient release in intestinal juice or simulated intestinal fluid can be stated to be pH 6.0.

EUDRAGIT® L 100-55 is a copolymer of 50% by weight ethyl acrylate and 50% by weight methacrylic acid. EUDRAGIT® L 30 D-55 is a dispersion comprising 30% by weight EUDRAGIT® L 100-55. The pH of the start of the specific active ingredient release in intestinal juice or simulated intestinal fluid can be stated to be pH 5.5.

Likewise suitable are anionic (meth)acrylate copolymers composed of 20 to 40% by weight methacrylic acid and 80 to 60% by weight methyl methacrylate (EUDRAGIT® S type). The pH of the start of the specific active ingredient release in intestinal juice or simulated intestinal fluid can be stated to be pH 7.0.

Suitable (meth)acrylate copolymers are those consisting of 10 to 30% by weight methyl methacrylate, 50 to 70% by weight methyl acrylate and 5 to 15% by weight methacrylic acid (EUDRAGIT® FS type). The pH at the start of the specific active ingredient release in intestinal juice or simulated intestinal fluid can be stated to be pH 7.0.

EUDRAGIT® FS is a copolymer of 25% by weight methyl meth-acrylate, 65% by weight methyl acrylate and 10% by weight methacrylic acid. EUDRAGIT® FS 30 D is a dispersion comprising 30% by weight EUDRAGIT® FS.

Additionally suitable is a copolymer composed of
20 to 34% by weight methacrylic acid and/or acrylic acid,
20 to 69% by weight methyl acrylate and
0 to 40% by weight ethyl acrylate and/or where appropriate
0 to 10% by weight further monomers capable of vinylic copolymerization,
   with the proviso that the glass transition temperature of the copolymer according to ISO 11357-2, subsection 3.3.3, is not more than 60° C. This (meth)acrylate copolymer is particularly suitable, because of its good elongation at break properties, for compressing pellets to tablets.

Additionally suitable is a copolymer composed of
20 to 33% by weight methacrylic acid and/or acrylic acid,
5 to 30% by weight methyl acrylate and
20 to 40% by weight ethyl acrylate and
more than 10 to 30% by weight butyl methacrylate and where appropriate 0 to 10% by weight further monomers capable of vinylic copolymerization, where the proportions of the monomers add up to 100% by weight,
   with the proviso that the glass transition temperature of the copolymer according to ISO 11357-2, subsection 3.3.3 (midpoint temperature T_{mg}), is 55 to 70° C. Copolymers of this type are particularly suitable, because of its good mechanical properties, for compressing pellets to tablets.

The abovementioned copolymer is composed in particular of free-radical polymerized units of

20 to 33, preferably 25 to 32, particularly preferably 28 to 31% by weight methacrylic acid or acrylic acid, with preference for methacrylic acid,
5 to 30, preferably 10 to 28, particularly preferably 15 to 25% by weight methyl acrylate,
20 to 40, preferably 25 to 35, particularly preferably 18 to 22% by weight ethyl acrylate, and
more than 10 to 30, preferably 15 to 25, particularly preferably 18 to 22% by weight butyl methacrylate,
where the monomer composition is chosen so that the glass transition temperature of the copolymer is from 55 to 70° C., preferably 59 to 66, particularly preferably 60 to 65° C.

Glass transition temperature means in this connection in particular the midpoint temperature T_{mg} according to ISO 11357-2, subsection 3.3.3. Measurement takes place without added plasticizer, with residual monomer contents (REMO) of less than 100 ppm, with a heating rate of 20° C./min and under a nitrogen atmosphere.

The copolymer preferably consists essentially to exclusively of 90, 95 or 99 to 100% by weight of the monomers methacrylic acid, methyl acrylate, ethyl acrylate and butyl methacrylate in the ranges of amounts indicated above.

However, it is possible, without this necessarily leading to an impairment of the essential properties, for small amounts in the range from 0 to 10, e.g. 1 to 5% by weight of further monomers capable of vinylic copolymerization additionally to be present, such as, for example, methyl methacrylate, butyl acrylate, hydroxyethyl methacrylate, vinylpyrrolidone, vinylmalonic acid, styrene, vinyl alcohol, vinyl acetate and/or derivatives thereof.

### Preparation of the Anionic (Meth)Acrylate Copolymers

The anionic (meth)acrylate copolymers can be prepared in a manner known per se by free-radical polymerization of the monomers (see, for example, EP 0 704 207 A2 and EP 0 704 208 A2). The copolymer according to the invention can be prepared in a manner known per se by free-radical emulsion polymerization in aqueous phase in the presence of, preferably, anionic emulsifiers, for example by the process described in DE-C 2 135 073.

The copolymer can be prepared by conventional processes of free-radical polymerization continuously or discontinuously by batch processes, for example emulsion polymerisation in the presence of free-radical forming initiators and, where appropriate, regulators to adjust the molecular weight undiluted, in solution, by bead polymerization or in emulsion. The average molecular weight Mw (weight average, determined for example by measuring the solution viscosity) may be for example in the range from 80 000 to 1 000 000 (g/mol). Emulsion polymerization in aqueous phase in the presence of water-soluble initiators and (preferably anionic) emulsifiers is preferred.

In the case of bulk polymerization, the copolymer can be obtained in solid form by crushing, extrusion, granulation or hot cut.

The (meth)acrylate copolymers are obtained in a manner known per se by free-radical bulk, solution, bead or emulsion polymerization. They must before processing be brought to the particle size range of the invention by suitable grinding, drying or spraying processes. This can take place by simple crushing of extruded and cooled pellets or hot cut.

The use of powders may be advantageous especially on mixture with other powders or liquids. Suitable equipments for producing powders are familiar to the skilled person, e.g. air jet mills, pinned disc mills, compartment mills. It is possible where appropriate to include appropriate sieving steps. A suitable mill for industrial large quantities is, for example, an opposed jet mill (Multi No. 4200) operated with a gauge pressure of about 6 bar.

### Additional Partial Neutralization

Compound (a) may be additionally partially neutralized by an alkaline agent, which is not a salt of the saturated monocarboxylic acids having 6 to 22 carbon atoms is selected from the group consisting of alkali metal salt and ammonium salt. The degree of such additional neutralisation may be around 1 to 20 or 1 to 10 mol %. This can be of advantage in cases where the suspension is not perfectly stable and tends to form sediment. This can be the case for instance when comparatively large amounts of pigments are added to the dispersion. A large of pigment can be for instance more than 100% by weight of the anionic polymeric material.

Bases suitable for such purposes are those expressly mentioned in EP 0 088 951 A2 or WO 2004/096185. The following are excluded in particular: Sodium hydroxide solution, potassium hydroxide solution (KOH), ammonium hydroxide or organic bases such as, for example, triethanolamine, sodium carbonate, potassium carbonate, sodium bicarbonate, trisodium phosphate, trisodium citrate or ammonia or physiologically tolerated amines such as tri-ethanolamine or tris(hydroxymethyl)aminomethane.

Further suitable cationic, organic bases are basic amino acids histidine, arginine and/or lysine.

### Adjustment of the Degree of Additional Partial Neutralization by Mixtures

Mixtures of anionic polymeric materials may also result in technical advantages in the adjustment of the degree of additional partial neutralization by an alkaline agent, which is not a salt of the saturated monocarboxylic acids having 6 to 22 carbon atoms. In a preferred embodiment of the invention for preparing the coating it is made use of mixtures of anionic (meth)acrylate copolymers differing in the degree of partial neutralization, consisting of free-radical polymerized units of 25 to 95% by weight C₁- to C₄-alkyl esters of acrylic or of methacrylic acid and 5 to 75% by weight (meth)acrylate monomers having an anionic group, wherein 1 up to 50 mol % of the contained anionic groups, as calculated average for the mixture, are neutralized by a base. It is possible for example to mix an anionic (meth)acrylate copolymer which is not partially neutralized and consists of free-radical polymerized units of 25 to 95% by weight C₁- to C₄-alkyl esters of acrylic or of methacrylic acid and 5 to 75% by weight (meth)acrylate monomers having an anionic group with a partially neutralized (meth)acrylate copolymer of the same monomer composition within the stated quantitative ranges so that 1 to 20 mol % of the contained anionic groups, as calculated average for the mixture, are neutralized. The mixture can be prepared for example by stirring a powder which has been obtained from a dispersion of a partially neutralized, anionic (meth)-acrylate copolymer, e.g. by spray drying or freeze drying, into a dispersion of an anionic (meth)acrylate copolymer which has not been partially neutralized.

### Powder Form

The composition according to the invention may be present as a (primary) powder which is a dry mixture of components (a) and (b) and optionally further pharmaceutical excipients. In this case the neutralisation process takes place not before the powder is dispersed in water to give a dispersion or a suspension. The composition according to the invention may be also present as a (secondary) powder form which is obtained from a dispersion of a dry mixture of components (a) and (b) and optionally further pharmaceutical excipients by freeze drying or spray drying. In this case the anionic material is already neutralized in the powder form.

### Coating

The Composition according to the invention may be present as a coated film of a gastric resistant, enteric-coated solid dosage form.

### Binding

The Composition according to the invention may be present as or used as a binding agent for the binding of pharmaceutically, nutraceutically or cosmetically active ingredients and optionally further excipients in a matrix structure which is a pharmaceutical, nutraceutical or cosmetical dosage form or a part of such a dosage form.

### Mixtures

The anionic polymeric material which has been partially neutralized according to the invention is further suitable for mixing with other pharmaceutically utilized polymers or copolymers in order to modify the properties thereof. This increases the scope for configuration by the skilled person when adjusting specifically modified release profiles. The proportion of other pharmaceutically utilized polymers or copolymers may be up to 40% by weight, up to 30% by weight, up to 20% by weight or up to 10% by weight, in relation to the anionic polymeric material. However it is also possible that essentially any or any other pharmaceutically utilized copolymers are included. The invention accordingly relates to a partially neutralized (meth)acrylate copolymer, characterized in that it is present in a mixture with copolymers of methyl methacrylate and/or ethyl acrylate and where appropriate less than 5% by weight methacrylic acid, copolymers of methyl methacrylate, butyl methacrylate and dimethylaminoethyl methacrylate, copolymers of methyl methacrylate, ethyl acrylate and trimethylammoniumethyl methacrylate, copolymers of methyl methacrylate and ethyl acrylate, polyvinylpyrrolidones (PVP), polyvinyl alcohols, polyvinyl alcohol-polyethylene glycol graft copolymers (Kollicoat®), starch and its derivatives, poly-vinyl acetate phthalate (PVAP, Coateric®), polyvinyl acetate (PVAc, Kollicoat), vinyl acetate-vinylpyrrolidone copolymer (Kollidon® VA64), polyethylene glycols having a molecular weight above 1000 (g/mol), chitosan, Na alginate, and/or a pectin. However any of the polymers or copolymers mentioned above may be present in the mixture or may be excluded from possible mixtures.

### Dispersions or Solutions

The non-neutralized or the partially neutralized (meth)acrylate copolymer may be for example in the form of an aqueous dispersion or solution with a solid content of 10 to 50 percent.

The non-neutralized or the partially neutralized (meth)acrylate copolymer may be in the form of a redispersible powder which has been obtained from a dispersion for example by spray drying.

### Dispersions/Partial Neutralization

The emulsion polymer is preferably produced and used in the form of an aqueous dispersion or solution with a solid content of 10 to 50 percent by weight, in particular 20 to 40% by weight. A solid content of 30% by weight is preferred as commercial form. For partial neutralization of the methacrylic acid units a base which is not a salt of a saturated, preferably unbranched, preferably unsubstituted, mono carboxylic acid (fatty acid) having 6 to 22, preferably 6 to 10 or 16 to 20 carbon atoms can be dispensed with for processing; it is, however, possible, for example to an extent of up to 5 or 10 mol %, if a stabilization or thickening of the coating agent dispersion is desirable. The weight-average size (radius) of the latex particles is normally 40 to 100 nm, preferably 50 to 70 nm, thus ensuring a viscosity below 1000 mPa·s which is favourable for processing techniques. The particle size can be determined by laser diffraction, e.g. using the Mastersizer 2000 (from Malvern Inc.).

In order to prepare a solution of the anionic copolymer it is normally necessary for the acidic groups to be partially or completely neutralized. The anionic copolymer may for example be stirred gradually in a final concentration of from 1 to 40% by weight into water and, during this, be partially or completely neutralized by adding a basic substance, liquid or solid, according to the invention such as, for example NaOH. It is also possible to employ a powder of the copolymer, to which a base has already been added during its preparation for the purpose of (partial) neutralization, so that the powder is already a (partially) neutralized polymer. The pH of the solution is normally above 4, e.g. in the range from 4 to about 8. It is also possible in this connection for batches of completely or partially neutralized dispersions to be mixed for example with non-neutralized dispersions and further processed in the manner described, i.e. use the mixture for coatings or initially freeze dry or spray dry to give a powder.

The dispersion may also for example be spray dried or freeze dried in a manner known per se and be provided in the form of a redispersible powder (see, for example, EP-A 0 262 326). Alternative processes are freeze drying or coagulation and squeezing out the water in an extruder with subsequent granulation (see, for example, EP-A 0 683 028).

Copolymer dispersions of spray-dried or freeze-dried and redispersed powders may exhibit increased shear stability. This is advantageous in particular for spray application. This advantage is strongly evident in particular when the copolymer present in the dispersion is partially neutralized to the extent of 2 to 10, preferably 5 to 7 mol-% (based on the acidic groups present in the copolymer). An anionic emulsifier is preferably present in an amount of 0.1 to 2% by weight.

### Pharmaceutical, Nutraceutical or Cosmetical Excipients

Composition according to the invention are further characterized in that pharmaceutically, nutraceutically or cosmetically acceptable additives or excipients, which may be selected from the group consisting of antioxidants, brighteners, flavouring agents, flow aids, fragrances, glidants, penetration-promoting agents, pigments, plasticizers, polymers, pore-forming agents or stabilizers may be included. In any case the excipients or additives that may be included are different from the components (a) and (b) according to the invention.

For instance up to 200%, up to 60%, up to 50%, up to 40%, up to 30%, up to 20% or up to 10% by weight of such excipients based on the total weight of the components (a) and (b) may be contained. However the composition according to the invention may as well contain any or essentially any pharmaceutical, nutraceutical or cosmetical excipients. Preferably no cationic (meth)acrylate copolymers that could interact with the anionic polymeric material (a) are contained. Thus the composition may essentially consist or consist to 100% of the components (a) and (b).

The term pharmaceutical, nutraceutical or cosmetical excipients is well known to the skilled person. Such excipients are customary in pharmacy but also in the field of nutraceuticals or cosmetics, occasionally also they are referred as customary additives. It is, of course, always necessary for all the excipients or customary additives employed to be toxicologically acceptable and usable in particular in food or in medicaments without a risk for customers or patients.

Although the requirements are usually higher in the pharmaceutical field there is a widely overlap of excipients used for pharmaceutical purposes and those used for nutraceutical purposes. Usually all pharmaceutical excipients may be used for nutraceutical purposes and at least a large number of nutraceutical excipients are allowed to be used for pharmaceutical purposes as well. Excipients may be are added to the formulation of the invention, preferably during the mixing of the powders production of the granules, coating of solids or patches or dispersing semi solids.

Pharmaceutical, nutraceutical or cosmetical excipients which are different from the components (a) and (b) may be contained for practical reasons, for instance to avoid stickiness or to add a colour. However these excipients usually do not contribute or do show any or almost no effect on the invention itself as claimed here.

Pharmaceutical, nutraceutical or cosmetical excipients do not contribute to the invention in a narrow sense which is based on the interaction of the components (a) and (b). Pharmaceutical, nutraceutical or cosmetical excipients which may have an essential adverse effect on the major beneficial effects of the present invention e.g. the preparation time or on the viscosity of the dispersion should be avoided and can be excluded.

Typical pharmaceutical, nutraceutical or cosmetical excipients which are different from the components (a) and (b) are familiar to those skilled in the art. Examples are antioxidants, brighteners, flavouring agents, flow aids, fragrances, glidants (release agents), penetration-promoting agents, pigments, plasticizers, pore-forming agents or stabilizers. They may be used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary as an additional control parameter.

### Plasticizers

Plasticizers achieve through physical interaction with a polymer a reduction in the glass transition temperature and promote film formation, depending on the added amount. Suitable substances usually have a molecular weight of between 100 and 20 000 and comprise one or more hydrophilic groups in the molecule, e.g. hydroxyl, ester or amino groups.

Examples of suitable plasticizers are alkyl citrates, glycerol esters, alkyl phthalates, alkyl sebacates, sucrose esters, sorbitan esters, diethyl sebacate, dibutyl sebacate, propylenglycol and polyethylene glycols 200 to 12 000. Preferred plasticizers are triethyl citrate (TEC), acetyl triethyl citrate (ATEC), diethyl sebacate and dibutyl sebacate (DBS). Mention should additionally be made of esters which are usually liquid at room temperature, such as citrates, phthalates, sebacates or castor oil. Esters of citric acid and sebacinic acid are preferably used.

Addition of the plasticizers to the formulation can be carried out in a known manner, directly, in aqueous solution or after thermal pre-treatment of the mixture. It is also possible to employ mixtures of plasticizers.

### Glidants/Release Agents/Detackifiers:

Glidants, release agents or detackifiers usually have lipophilic properties and are usually added to spray suspensions. They prevent agglomeration of cores during film formation. There are preferably used talc, Mg or Ca stearate, ground silica, kaolin or nonionic emulsifiers with an HLB value of between 2 and 8. Standard proportions for use of release agents in the inventive coating and binding agents range between 0.5 and 70 wt % relative to the components (a) and (b).

### Fillers

Standard fillers are usually added to the inventive formulation during processing to coating and binding agents. The quantities introduced and the use of standard fillers in pharmaceutical coatings or overlayers is familiar to those skilled in the art. Examples of standard fillers are release agents, pigments, stabilizers, antioxidants, pore-forming agents, penetration-promoting agents, brighteners, fragrances or flavouring agents. They are used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary as an additional control parameter.

### Glidants (Release Agents):

Glidants or release agents usually have lipophilic properties and are usually added to spray suspensions. They prevent agglomeration of cores during film formation. There are preferably used talc, Mg or Ca stearate, ground silica, kaolin or nonionic emulsifiers with an HLB value of between 2 and 8. Standard proportions for use of release agents in the inventive coating and binding agents range between 0.5 and 100 wt % relative to copolymer.

In a particularly advantageous embodiment, the release agent is added in concentrated form as the outer layer. Application takes place in the form of powder or by spraying from aqueous suspension with 5 to 30% solid content. The necessary concentration is lower than for incorporation into the polymer layer and amounts to 0.1 to 2% relative to the weight of the pharmaceutical form.

### Pigments:

Only rarely is the pigment added in soluble form. As a rule, aluminum oxide or iron oxide pigments are used in dispersed form. Titanium dioxide is used as a whitening pigment. Standard proportions for use of pigments in the inventive coating and binding agents range between 10 and 2000 wt-% relative to the mixture of components (a) and (b)

Because of the high pigment-binding capacity, however, proportions as high as 100 wt % can also be processed.

In a particularly advantageous embodiment, the pigment is used directly in concentrated form as the outer layer. Application takes place in the form of powder or by spraying from aqueous suspension with 5 to 35% solid content. The necessary concentration is lower than for incorporation into the polymer layer and amounts to 0.1 to 2% relative to the weight of the pharmaceutical form.

In principle, all substances used must of course be toxicologically safe and be used in pharmaceuticals without risk for patients.

### The Preparation Process

Components (a) and (b) are intermixed with each other with or without addition of water, and in the latter case followed by subsequent addition of water. The coating of the pharmaceutical form for example is prepared by spraying. In this connection transformation of the composition into a film (coating) is the prerequisite for the functional effect in pharmaceutical forms.

According to the invention the components (a) and (b), preferably component (a) may be processed as a solution in organic solvents. Suitable solvents may be liquid alcohols, esters or ketons, such as methanol, ethanol, propanol, isopropanol, acetone or ethylacetat. The solvent may be evaporate after intermixing.

### Powder Mixture Process

Components (a) and (b) are intermixed with each other in a powdery stage by using mixer equipment. Powdery stage can be defined in that the particle of components may have an average particle size of less than 1 mm, preferably of less than 0.5 mm, especially of 100 μm or less, preferably in the range 10 to 100 μm. The process of powder mixing is well known to a skilled person. The average particle size may be determined by sieving techniques or by laser diffraction methods.

### Dry Granulation Process

Components (a) and (b) are intermixed with each other in a form of granulates by using a mixer equipment. Granulates may have an average particle size of 1 mm or more, preferably in the range of 1 to 5 mm.

### Wet Granulation Process

Powders or granules of components (a) and (b) are intermixed with each other in a wet stage by wetting the powders or granulates with water or organic solvents and then using a mixer or kneading equipment. Wet stage shall mean that there is a wet mass than can be manually kneaded with a water content for instance in the range 10 to 100% by weight. After wetting and mixing respectively kneading the wet mass is dried and then again commuted to granules or powders. The process of wet granulation is well known to a skilled person. Polymer solutions in organic solvents like methanol, ethanol, isopropanol, ethyl acetate or acetone may also be used in the wet granulation process. The organic solvents may optionally contain up to 50% (v/v) of water.

### Melt Granulation Process

Powders or granules of components (a) and (b) are intermixed with each other usually without the addition of solvents at elevated temperatures where at least the copolymer is in a molten stage. This can be performed in a heated mixer or in an extruder, preferably in a twin screw extruder. After mixing the molten mass is cooled and then again commuted to granules or to powders. The process of melt granulation is well known to a skilled person.

### Dispersion or Solution Process

The components (a) and (b) are added to the aqueous dispersing or solution agent, preferably purified water, as powder mixtures, granules or single one after another while gentle stirring with a conventional stirrer at room temperature. Advantageously, according to this invention, the need of a high shear mixer or specific disperser will not be necessary. Additionally, the heating of the suspension will be not necessary. After stirring less than 5 hours dispersions or solutions are formed being able to be sprayed in coating or granulation processes and/or to form films after drying. The dispersion or solution may have a total content of solids less than 35% by weight, preferably less than 25% by weight and pH-values from 3 to 8. The pH values of a dispersion or solution may in the range from 4 to 7, preferably from 5 to 6.

### Dispersion Preparation Time

The dispersion preparation time can for instance be observed and determined by polarization microscopy. The time when the dry powdery or granulate mixture is stirred into the water is defined as starting point. The dispersing aqueous mixture is further stirred at room temperature (ca. 22° C.). At the beginning there is a turbid dispersion, that becomes first white and then more and more clear during stirring. Drops of the dispersing aqueous mixture are then taken every 10 minutes and observed under a polarization microscope with a magnification of 100-fold with the support of a phase filter. The time point when no or almost no particles (at least less than ten particles in the view field) are observed in the fluid of such a drop under the microscope is taken as end point of the dispersion process. The accuracy of this determination method is in most cases sufficient to differ the preparation times of the different dispersion preparations apart from each other. The inventive composition may be characterized by a dispersion preparation time of 4 hours or less, preferably 2.5 hours or less most preferred 1.5 hours or less, starting respectively measured from the stirring the dry powdery or granulate mixture into water at room temperature, further stirring and thereby dissolving the components to a clear dispersion or solution respectively.

### Practical Applications

Dispersions according to this invention may be used in granulation or coating process in the development and manufacturing of nutrition supplements, nutraceuticals, cosmetics, cosmeceuticals, pharmaceutical intermediates or pharmaceuticals. Due to the physicochemical properties of the polymer, which are maintained in the dispersed compounds of this invention, functions such as coloring, taste masking, moisture protection, light protection, odor masking or eased swelling are introduced into the final dosage form.

Application procedures and processes known to the skilled person and published for example in:
G. Cole, J. Hogan, M. Aulton, Pharmaceutical coating Technology Taylor & Francis,
K. H. Bauer, K. Lehmann, H. P. Osterwald, G. Rothgang, “Coated Dosage Forms”, CRC Press 1998
Pharmaceutical Manufacturing Encyclopedia, William Andrew Publishing; Third Edition, 2005
Encyclopedia of Pharmaceutical Technology, Third Edition, Informa Healthcare, 2006
J. W. McGinity, L. A. Felton, aqueous Polymeric Coatings for Pharmaceutical Dosage Forms, Third Edition, Informa Healthcare, 2008

Transformation to film takes place by input of energy, regardless of the application process. This can be accomplished by convection (heat), radiation (infrared or microwave) or conduction. Water used as suspension agent for application then evaporates. If necessary, a vacuum can also be employed to accelerate evaporation. The temperature required for transformation to film depends on the combination of components used.

### Use of the Partially Neutralized (Meth)Acrylate Copolymers

The partially neutralized anionic (meth)acrylate copolymer may be used as a coating agent for preparing the for a pharmaceutical form which, in the USP 28 release test after 2 hours at pH 1.2 and a subsequent change in the buffer to the pH of the start of active ingredient release, releases 90%, preferably 95 or 100% of the contained active ingredient within a specified time.

The USP 28 release test, in particular by USP 28 <711> paddle method (=Apparatus 2), is sufficiently well known to the skilled person.

The typical test procedure is as follows:
1. The vessels of the release apparatus are each charged with 900 ml of 0.1M-HCl (pH 1.2) and the temperature of the waterbath is adjusted to 37±0.5° C.
2. The paddle stirrer is switched on with a rotation rate of 50 rpm.
3. 1 tablet or a certain amount of pellets containing a comparable amount of active ingredient as 1 tablet is put into each vessel of the apparatus. Care is taken that there are no air bubbles on the pellet or tablet surface.
4. After 120 min, tablets or pellets are removed from the acid, and put into 900 ml phosphate buffer solution with pH values of pH 5.5; 5.6; 5.8; 6.0 or 7.0.
5. Determination of the percentage of active ingredient release as a function of time, depending on the active ingredient, e.g. by photometry at 271 nm in the case of theophylline, or 247 nm in the case of prednisolone, in the circulating method.

### Pharmaceutical or Nutraceutical Dosage Form

In a preferred embodiment of the invention a pharmaceutical form is comprising a core having an active pharmaceutical ingredient and comprising a polymer coating of a partially neutralized (meth)acrylate copolymer. The pharmaceutical form may preferably comprise a polymer coating with NaOH as neutralizing agent in combination with 0 to 70% by weight of a plasticizer.

The corresponding pharmaceutical form may be for example in the form of a multiparticulate pharmaceutical form, pellet-containing tablets, minitablets, capsules, sachets, effervescent tablets or reconstitutable powders.

### Process for Producing a Pharmaceutical Form

The invention further relates to a process for producing the pharmaceutical form according to the invention in a manner known per se by pharmaceutically customary processes such as direct compression, compression of dry, wet or sintered granules, extrusion and subsequent rounding off, wet or dry granulation or direct pelleting or by binding powders (powder layering) onto active ingredient-free beads or neutral cores (nonpareilles) or active ingredient-containing particles and by applying the polymer coating in a spray process or by fluidized bed granulation.

### Production of Multiparticulate Pharmaceutical Forms

The invention is suitable in particular for producing multiparticulate pharmaceutical forms, because the copolymer according to the invention withstands the high pressures in the compression of the pellets with the filler.

The production of multiparticulate pharmaceutical forms by compression of a pharmaceutically usual binder with active ingredient-containing particles is described in detail for example Beckert et al. (1996), “Compression of enteric-coated pellets to disintegrating tablets”, International Journal of Pharmaceutics 143, pp. 13-23, and in WO 96/01624.

Active ingredient-containing pellets can be produced by applying active ingredient by means of a layering process. For this purpose, active ingredient is homogenized together with further excipients (release agent, where appropriate plasticizer) and dissolved or suspended in a binder. The liquid can be applied by means of a fluidized bed process to placebo pellets or other suitable carrier materials, with evaporation of the solvent or suspending agent (literature: International Journal of Pharmaceutics 143, pp. 13-23). The production process may be followed by a drying step. The active ingredient can be applied in a plurality of layers.

Some active ingredients, e.g. acetylsalicylic acid, are commercially available in the form of active ingredient crystals and can be employed in this form instead of active ingredient-containing pellets.

Film coatings on active ingredient-containing pellets are normally applied in fluidized bed apparatuses. Formulation examples are mentioned in this application. Film formers are normally mixed with plasticizers and release agents by a suitable process. It is possible in this case for the film formers to be in the form of a solution or suspension. The excipients for the film formation may likewise be dissolved or suspended. Organic or aqueous solvents or dispersants can be used. It is additionally possible to use stabilizers to stabilize the dispersion (for example: Tween 80 or other suitable emulsifiers or stabilizers).

Examples of release agents are glycerol monostearate or other suitable fatty acid derivatives, silica derivatives or talc. Examples of plasticizers are propylene glycol, phthalates, polyethylene glycols, sebacates or citrates, and other substances mentioned in the literature.

Mixtures for producing tablets from coated particles are prepared by mixing the pellets with suitable binders for tableting, if necessary adding disintegration-promoting substances and if necessary adding lubricants. The mixing can take place in suitable machines. Unsuitable mixers are those leading to damage to the coated particles, e.g. ploughshare mixers. A specific sequence of addition of the excipients to the coated particles may be necessary to achieve suitable short disintegration times. It is possible by premixing with the coated particles with the lubricant or mould release agent magnesium stearate for its surface to be rendered hydrophobic and thus for adhesion to be avoided.

Mixtures suitable for tableting normally comprise 3 to 15% by weight of a disintegration aid, e.g. starch or crosslinked polyvinyl pyrrolidone and, for example, 0.1 to 1% by weight of a lubricant and mould release agent such as magnesium stearate. The binder content is determined by the required proportion of coated particles.

Examples of typical binders are Cellactose®, microcrystalline cellulose, calcium phosphates, Ludipress®, lactose or other suitable sugars, calcium sulphates or starch derivatives. Substances of low apparent density are preferred.

Typical disintegration aids (disintegrants) are crosslinked starch or cellulose derivatives, and crosslinked polyvinylpyrrolidone. Cellulose derivatives are likewise suitable. The use of disintegration aids can be dispensed with through selection of a suitable binder.

Typical lubricants and mould release agents are magnesium stearates or other suitable salts of fatty acids or substances mentioned in the literature for this purpose (e.g. lauric acid, calcium stearate, talc, etc.). The use of a lubricant and mould release agent in the mixture can be dispensed with on use of suitable machines (e.g. tablet press with external lubrication), or suitable formulations.

A flow-improving aid can be added where appropriate to the mixture (e.g. colloidal silica derivatives, talc etc.).

The tableting can take place on conventional tablet presses, eccentric or rotary tablet presses, with compressive forces in the range from 5 to 40 kN, preferably 10-20 kN. The tablet presses may be equipped with systems for external lubrication. Special systems for die filling which avoid die filling by means of impeller paddles are employed where appropriate.

### Further Processes for Producing the Pharmaceutical Form According to the Invention

Preferably the application as a coating takes place by spray application of aqueous dispersions. Alternatively the application as a coating may take place by spray application of a solvent based liquid or by direct powder application or powder coating. The crucial factor for the implementation is that uniform, pore-free coatings result.

For prior art application processes see, for example, Bauer, Lehmann, Osterwald, Rothgang, “Überzogene Arzneiformen” Wissenschaftliche Verlagsgesellschaft mbH, Stuttgart, Chapter 7, pp. 165-196

Relevant properties, required tests and specifications for the application are listed in pharmacopoeias.

Details are to be found in the customary textbooks, for example:
Voigt, R. (1984): Lehrbuch der pharmazeutischen Technologie; Verlag Chemie Weinheim—Deerfield Beach/Florida—Basel.
Sucker, H., Fuchs, P., Speiser, P.: Pharmazeutische Technologie, Georg Thieme Verlag Stuttgart (1991), especially Chapters 15 and 16, pp. 626-642.
Gennaro, A. R. (editor), Remington's Pharmaceutical Sciences, Mack Publishing Co., Easton Pa. (1985), Chapter 88, pp. 1567-1573.
List, P. H. (1982): Arzneiformenlehre, Wissenschaftliche Verlagsgesellschaft mbH, Stuttgart.

The present invention will be further explained in more detail by the following examples, which are understood not to limit the scope of the invention in any way.

### Nutraceuticals

Nutraceuticals can be defined as extracts of foods claimed to have medical effects on human health. The nutraceutical is usual contained in a medical format such as capsule, tablet or powder in a prescribed dose. Examples for nutraceuticals are resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals is used as synonym for nutraceuticals.

### Cosmetics

Cosmetics are substances used to enhance or protect the appearance or odor of the human body. Cosmetics include skin-care creams, lotions, powders, perfumes, lipsticks, fingernail and toe nail polish, eye and facial makeup, permanent waves, colored contact lenses, hair colors, hair sprays and gels, deodorants, baby products, bath oils, bubble baths, bath salts, butters and many other types of products. Their use is widespread, especially among women in Western countries. A subset of cosmetics is called “make-up,” which refers primarily to colored products intended to alter the user's appearance. Many manufacturers distinguish between decorative cosmetics and care cosmetics.

### Use

The invention discloses the use of the composition as a coating or binding agent for the spray coating or binding of pharmaceutical, nutraceutical or cosmetical compositions. Preferred active ingredient containing compositions may be in the form of pellets, granules, minitablets, tablets or capsules or nutraceutical compositions or cosmetical compositions. The use as a coating solution shall include the use as a subcoat or a topcoat in combination with other coatings.

### EXAMPLES

The following copolymers were used in the Examples.

### Copolymer 1:

Obtained from 50 weight percent of ethyl acrylate and 50 weight percent methacrylic acid (EUDRAGIT® L 100-55) used without neutralization. EUDRAGIT® L30D-55 is a 30% by weight aqueous dispersion of EUDRAGIT® L 100-55.

### Copolymer 2:

Obtained from 50 weight percent of methyl methacrylate and 50 weight percent methacrylic acid (EUDRAGIT® L100) used without neutralization.

### Copolymer 3:

Obtained from 70 weight percent of methyl methacrylate and 30 weight percent methacrylic acid (EUDRAGIT® S100) used without neutralization.

### Summary Table for the Examples

| | | | | Salt/acid | | | | |
|---|---|---|---|---|---|---|---|---|
| | | | | Na—C₁₈ | Na—C₁₀ | Na—C₈ | | |
| | Copolymer | | | Sodium- | Sodium | Sodium- | divergent | |
| Ex. Nr | 1 | 2 | 3 | Stearate | Caprarte | Caprylate | feature | mol % |
| 1 | + | | | + | | | | 9 |
| 2 | + | | | | + | | | 9 |
| 3 | + | | | + | | | | 9 |
| 4 | + | | | + | | | | 7 |
| 5 | + | | | + | | | | 7 |
| 6 | + | | | + | | | | 9 |
| 7 | | + | | | + | | | |
| 8 | | + | | | + | | | |
| 9 | | + | | | + | | | 9 |
| 10 | + | | | + | | | | 9 |
| 11 | + | | | + | | | NaOH + stearic | 9 |
| | | | | | | | acid | |
| 12 | + | | | + | | | KOH + stearic | 9 |
| | | | | | | | acid | |
| 13 | | | + | | + | | | 9 |
| 14 | d | | | + | | | | 9 |
| 15 | d | | | | + | | | 9 |
| 16 | d | | | | | + | | |
| 17 | | | + | | + | | | 20 |
| 18 | | | + | | + | | | 9 |
| 19 | | | + | + | | | | 15 |
| 20 | | | + | + | | | | 15 |
| C21 | + | | | | | | Na-propaonate | 9 |
| C22 | + | | | | | | sodium citrate | 3 |
| C23 | + | | | | | | sodium citrate | 9 |
| 24 | + | | | | | | Na-behenate | 7 |
| 25 | d | | | + | | | | 9 |
| 26 | d | | | | | + | | 11 |
| 27 | d | | | | | + | | 5 |
| 28 | d | | | | | + | | 5 |
| 29 | d | | | | | + | | 5 |
| 30 | + | | | | | | | 9 |
| C31 | | | + | | | + | | 55 |
| 32 | | | | | | + | Copolymer HP55 | 50 |
| C33 | + | | | | | + | | 2 |
| 34 | + | | | | | | Na-2-hydroxy- | 9 |
| | | | | | | | octonate | |
| C35 | + | | | | | | Capric acid | 9 |
| 36 | + | | | + | | | | 9 |
| 37 | | | | | | + | | 9 |

Abbreviations:
Ex. Nr. = Example number; numbers beginning with a “C” are comparative examples
d = copolymer is used in form dispersion form (EUDRAGIT ® L30D-55)
mol. % = degree of neutralisation of the anionic groups of the copolymer in mol-%
Copolymer 1 = EUDRAGIT ® L100-55; Copolymer 2 = EUDRAGIT ® L;
Copolymer 3 = EUDRAGIT ® S, HP55 = hydroxypropyl methyl cellulose phthalate

### Example 1

7.7 g sodium stearate was added to 80.0 g deionized water and heated to 52° C. under simple stirring. A high viscous aqueous suspension was formed. 50.0 g Copolymer 1 was suspended in 150 g deionized water and stirred for 10 minutes using a dissolver stirrer at a speed of 550 rpm. Sodium stearate suspension was added to the copolymer suspension and stirred for further 60 minutes at room temperature until a dispersion of low viscosity was obtained. The degree of neutralization of the anionic polymer was about 9 mol %. After drying a sample at room temperature, a solid, brittle, and clear film was formed, indicating film forming functionality. 0.3 g of the film was stable in 0.1 M HCl (pH 1.2) over 120 min according the USP 28 <711> paddle method (Apparatus 2) at 50 rpm.

### Example 2

4.9 g sodium caprate was added to 80.0 g demineralized water and heated to 52° C. under simple stirring. A low viscous colloidal solution was formed. 50.0 g Copolymer 1 was suspended in 140 g deionized water and stirred for 10 minutes using a dissolver stirrer at a speed of 550 rpm. Sodium caprate suspension was added to the copolymer suspension and stirred for further 60 minutes at room temperature until a dispersion of low viscosity was obtained. The degree of neutralization of the anionic polymer was about 9 mol %. After drying a sample at room temperature, a solid, brittle, and clear film was formed, indicating film forming functionality. 0.3 g of the film was stable in 0.1 M HCl (pH 1.2) over 120 min according the USP 28 <711> paddle method (Apparatus 2) at 50 rpm.

### Example 3

10.0 g EUDRAGIT® L 100-55 and 1.55 sodium stearate were mixed in powder form for 15 minutes using a Turbular shaker-mixer. This mixture was added in small amounts to 65.5 g demineralized water while stirring at room temperature (25° C.). The powder mixture readily dispersed when added to the water. The degree of neutralization of the anionic polymer was about 9 mol %. 0.3 g of the film was stable in 0.1 M HCl (pH 1.2) over 120 min according the USP 28 <711> paddle method (Apparatus 2) at 50 rpm.

### Example 4

40.0 g EUDRAGIT® L 100-55, 4.65 sodium stearate, 15.0 g talc and 3.0 g pigment Candurin Red were mixed in powder form for 15 minutes and subsequently subjected to a sieving step using a 0.4 mm sieve in order to obtain a homogenous powder mixture consisting of:

63.4 weight percent EUDRAGIT® L 100-55;
23.9 weight percent talc,
7.5 weight percent sodium stearate, and
4.8 pigment Candurin Red Lustre.
The amount of sodium stearate in the composition corresponds to about 7 mol percent of the amount of anionic groups in the polymeric material

### Example 5

25.0 g of the powder mixture of example 4 were added in small amounts to 141.67 g demineralized water and stirred at 550 rotations per minute for 10 minutes using a dissolver stirrer. Once the powder mixture was completely added to the water the mixture was further stirred for 1 h at room temperature. After 1 hour the mixture was completely dissolved. The dispersion obtained is forming a red and flexible to brittle film when dried. 0.3 g of the film was stable in 0.1 M HCl (pH 1.2) over 120 min according the USP 28 <711> paddle method (Apparatus 2) at 50 rpm. The composition of the dispersion was as follows:

15.95 g EUDRAGIT® L 100-55 (9.6 weight percent);
5.98 g talc (3.6 weight percent),
1.85 g sodium stearate (1.1 weight percent),
1.20 g pigment Candurin Red Lustre (0.7 weight percent), and
141.67 g demineralized water (85 weight percent).

### Example 6

### EUDRAGIT® L 100-55 Spray-Coating and Release Tests

100 g theophylline pellets (1.0 to 1.25 mm in size) were coated in a Hüttlin Mycrolab device using the dispersions prepared according to example 5. Table 1 summarizes the coating conditions for theophylline pellets.

TABLE 1
coating conditions for theophylline pellets.

| Formulation | Eudragit ® L 100-55 |
|---|---|
| inlet temperature (° C.) | 70-75 |
| bed temperature (° C.) | 49-51 |
| outlet temperature (° C.) | 32-36 |
| air flow rate (m³/h) | 16-17 m³/h |
| Nozzle bore (mm) | 0.8 |
| atomizing pressure (bar) | 0.8 |
| spray rate (ml/min) | 1.3-2.2 |

The spraying time for 10 percent weight gain based on polymer weight was 56 minutes (116.9 g of the dispersions obtained according to example 5). The spraying time for 15 percent weight gain based on polymer weight was 70 minutes. The coated pellets obtained by the spraying process were tested for release of theophylline.

The dissolution test for coated pellets comprising as active ingredient theophylline, were carried out using BP Method II paddle apparatus (Model PTWS, Pharmatest, Hainburg, Germany). The volume of the dissolution media was 900 ml maintained at 37±0.5° C. and a paddle speed of 100 rpm was employed. The amount of theophylline released from the coated tablets or pellets was determined by UV spectrophotometer at 271 nm for theophylline. The pellets were placed for 120 min into 0.1N HCl, and subsequently into phosphate buffer pH 6.0. Table 2 summarizes the release of theophylline.

Table 2 shows the dissolution profiles of EUDRAGIT® L 100-55 theophylline pellets in 0.1N HCl for 2 h and subsequent pH 6.0 phosphate buffer.

| time [min] | minimum (%) | average (%) | maximum (%) |
|---|---|---|---|
| 0 | 0.00 | 0.01 | 0.02 |
| 15 | 0.15 | 0.2 | 0.23 |
| 30 | 0.45 | 0.48 | 0.52 |
| 45 | 0.7 | 0.72 | 0.72 |
| 60 | 0.88 | 0.92 | 0.98 |
| 90 | 1.29 | 1.33 | 1.38 |
| 120 | 1.71 | 1.78 | 1.84 |
| 125 | 2.36 | 2.41 | 2.49 |
| 130 | 27.72 | 28.51 | 29.09 |
| 135 | 62.95 | 63.03 | 63.17 |
| 140 | 83.47 | 83.52 | 83.57 |
| 145 | 94.3 | 94.52 | 94.65 |
| 150 | 98.98 | 99.32 | 99.56 |
| 155 | 99.81 | 99.91 | 99.95 |
| 160 | 99.75 | 99.85 | 99.93 |
| 165 | 99.97 | 100.08 | 100.14 |
| 180 | 99.92 | 100.00 | 100.05 |
| 210 | 100.05 | 100.14 | 100.31 |

### Example 7

30.0 g EUDRAGIT® L 100 were mixed with 2.95 sodium caprate, 15.0 g talc and 3.0 g pigment Candurin Red in powder form for 15 minutes in a Turbular shaker-mixer in order to obtain a homogenous powder mixture consisting of:

58.88 weight percent Eudragit® L 100;
29.44 weight percent talc,
5.79 weight percent sodium caprate, and
5.89 pigment Candurin Red Lustre.
The amount of sodium caprate in the composition corresponds to about 9 mol percent of the amount of anionic groups in the polymeric material

### Example 8

25.0 g of the powder mixture of example 7 were added in small amounts to 141.67 g demineralized water and stirred at 550 per minute for 10 minutes using a dissolver stirrer. Once the powder mixture was added to the water completely the mixture was further stirred for 1 h at room temperature at 1100 rpm. After 1 hour the mixture was completely dissolved. There have not been observed any polymer grains when checking the obtained dispersion by microscope. The dispersion obtained is forming a brittle, opaque to red film when dried. 0.3 g of the film was stable in 0.1 M HCl (pH 1.2) over 120 min according the USP 28 <711> paddle method (Apparatus 2) at 50 rpm. The composition of the dispersion is as follows:

14.72 g Eudragit® L 100 (8.83 weight percent);
7.36 g talc (4.42 weight percent),
1.45 g sodium caprate (0.87 weight percent),
1.47 g pigment Candurin Red Lustre (0.88 weight percent), and
141.67 g demineralized water (85 weight percent).

### Example 9

25.0 g EUDRAGIT® L 100 was added with 100 g demineralized water and stirred for 5 minutes. Then 5.45 g sodium caprate was disslolved in 22.0 g demineralized water and added to the EUDRAGIT® L 100 mixture. Within 5 minutes the polymer is completely dispersed. The degree of neutralization of the anionic polymer was about 20 mol %. A part of the dispersion was dried at room temperature wherein a brittle and cloudy film was obtained. Another part of the dispersion was dried at 40° C. and a brittle, clear and shining film was obtained. 0.3 g of the film was stable in 0.1 M HCl (pH 1.2) over 120 min according the USP 28 <711> paddle method (Apparatus 2) at 50 rpm.

### Example 10

30.0 g EUDRAGIT® L 100-55 and 4.65 g sodium stearate were mixed in powder form for 15 minutes using a Turbular shaker-mixer. 5.0 g of this powder mixture was mixed with 0.43 triethylcitrate (TEC) and added with 20.0 g demineralized water while stirring at room temperature. The degree of neutralization of the anionic polymer was about 9 mol %. When the powder mixture was completely dissolved the dispersion could be dried to a clear to cloudy flexible film. 0.3 g of the film was stable in 0.1 M HCl (pH 1.2) over 120 min according the USP 28 <711> paddle method (Apparatus 2) at 50 rpm.

### Example 11

a) 12.0 g EUDRAGIT® L 100-55 was added to 24 g demineralized water and stirred at room temperature, b) 6.34 g 1 N NaOH was added with 79.0 g demineralized water and 1.8 g stearic acid and dissolved at 58° C. After cooling to 40° C. solutions a) and b) were mixed and a dispersion with low viscosity was obtained in which the polymer was completely dispersed. The degree of neutralization of the anionic polymer was about 9 mol %. The dispersion obtained dried to a solid and clear to cloudy film at room temperature. 0.3 g of the film was stable in 0.1 M HCl (pH 1.2) over 120 min according the USP 28 <711> paddle method (Apparatus 2) at 50 rpm.

### Example 12

a) 12.0 g EUDRAGIT® L 100-55 was added to 50 g demineralized water and stirred at room temperature, b) 30.0 g demineralized water was added with 6.34 g 1 N KOH and 1.8 g stearic acid and dissolved at 58° C. After cooling to 50° C. solutions a) and b) were mixed and stirred for 1 h. KOH and stearic acid react in situ to form sodium stearate. The degree of neutralization of the anionic polymer was about 9 mol %. A dispersion was obtained in which the polymer was completely dispersed. The dispersion obtained dried to a solid and clear to cloudy film at room temperature. 0.3 g of the film was stable in 0.1 M HCl (pH 1.2) over 120 min according the USP 28 <711> paddle method (Apparatus 2) at 50 rpm.

### Example 13

25.0 g EUDRAGIT® S 100 was added to 100 g demineralized water and stirred using a dissolver stirrer. Subsequently a solution containing 1.48 g sodium caprate in 25.05 g demineralized water was added. The degree of neutralization of the anionic polymer was about 9 mol %. The dispersion obtained dried at room temperature to form an opaque brittle film. 0.3 g of the film was stable in 0.1 M HCl (pH 1.2) over 120 min according the USP 28 <711> paddle method (Apparatus 2) at 50 rpm.

### Example 14

a) 120.0 g of a EUDRAGIT® L 30-D55 dispersion was diluted with 16.0 g demineralized water. b) 5.57 g sodium stearate was dissolved with 55.7 g demineralized water at 60° C. and added with further 16.0 g demineralized cold water in order to cool the solution to 50° C. The solution was added to solution a) under stirring using a dissolver stirrer at 600 rpm. After a transient increase of the viscosity for 2 min the solution was stirred at 1000 rpm for 30 min in order to obtain a dispersion. The degree of neutralization of the anionic polymer was about 9 mol %. When subjecting the disperision to a sieving step using a 0.315 mm sieve 0.05 weight percent retentate is obtained. The dispersion obtained dried at room temperature to form an opaque brittle film. 0.3 g of the film was stable in 0.1 M HCl (pH 1.2) over 120 min according the USP 28 <711> paddle method (Apparatus 2) at 50 rpm.

### Example 15

a) 120.0 g of EUDRAGIT® L 30-D55 dispersion was diluted with 19.0 g demineralized water. b) 3.53 g sodium caprate was dissolved in 55.3 g demineralized water at room temperature and added to solution a) under stirring using a dissolver stirrer at 600 rpm. After a transient increase of the viscosity for 2 min the solution is stirred at 1000 rpm for 30 min in order to obtain a dispersion. The degree of neutralization of the anionic polymer was about 9 mol %. When subjecting the disperision to a sieving step using a 0.315 mm sieve only foam is retained. The dispersion obtained dried at room temperature to form an opaque brittle film. 0.3 g of the film was stable in 0.1 M HCl (pH 1.2) over 120 min according the USP 28 <711> paddle method (Apparatus 2) at 50 rpm.

### Example 16

a) 120.0 g of an EUDRAGIT® L 30-D55 dispersion was diluted with 20.0 g demineralized water. b) 3.02 g sodium caprylate was dissolved in 52.3 g demineralized water at room temperature and added to solution a) under stirring using a dissolver stirrer at 600 rpm. After a transient increase of the viscosity for 2 min the solution is stirred at 1000 rpm for 30 min in order to obtain a dispersion. The degree of neutralization of the anionic polymer was about 9 mol %. When subjecting the dispersion to a sieving step using a 0.315 mm sieve there remains no retentate. The dispersion is drying to a flexible, clear and glossy film. 0.3 g of the film was stable in 0.1 M HCl (pH 1.2) over 120 min according the USP 28 <711> paddle method (Apparatus 2) at 50 rpm. When spray coating theophylline pellets using 10% weight gain nearly the same gastric resistant and enteric coating characteristics as in example 6 were obtained.

### Example 17

60.0 g of the dispersion obtained in example 13 were added with 0.716 sodium caprate in order that 20% of the anionic groups participated in the reaction. The degree of neutralization of the anionic polymer was about 20 mol %. The dispersion dried as described in example 13 at room temperature to a white/opaque brittle layer. 0.3 g of the film was stable in 0.1 M HCl (pH 1.2) over 120 min according the USP 28 <711> paddle method (Apparatus 2) at 50 rpm. The addition of plasticiser (e.g. 50 weight percent triethylcitrate based on polymer weight) can significantly improve the properties of the film.

### Example 18

40.0 g of the dispersion obtained in example 13 were added with 1.32 g propylene glycol and stirred. The degree of neutralization of the anionic polymer was about 9 mol %. The dispersion dried to a clear, glossy to opaque, brittle film. 0.3 g of the film was stable in 0.1 M HCl (pH 1.2) over 120 min according the USP 28 <711> paddle method (Apparatus 2) at 50 rpm.

### Example 19

30.0 g Eudragit® S 100, 10.0 g PEG 6000, 3.0 g talc, 3.0 g Pigment Candurin Red Lustre and 4.67 g sodium stearate (corresponding to 15% partial neutralization of the anionic groups) were mixed in a Turbular shaker-mixer for 15 min, in order to obtain a red homogenous powder mixture.

### Example 20

20.0 g of the mixture obtained in example 19 were added to 80.0 g demineralized water and stirred at room temperature and a dispersion is obtained which dried to a white/opaque brittle layer. 0.3 g of the film was stable in 0.1 M HCl (pH 1.2) over 120 min according the USP 28 <711> paddle method (Apparatus 2) at 50 rpm.

### Comparative example 21

2.43 g sodium propanoate (3 carbon atoms) was added to 80.0 g deionized water under simple stirring. 50.0 g Copolymer 1 was suspended in 150 g deionized water and stirred for 10 minutes using a dissolver stirrer at a speed of 550 rpm. Sodium propanate solution was added to the copolymer suspension and stirred for further 24 hours at room temperature and no dispersion was obtained. The degree of neutralization of the anionic polymer was about 9 mol %. After drying a sample at room temperature, a solid, brittle, and opaque inhomogenous artefact was formed. 0.3 g of the artefact was not stable in 0.1 M HCl (pH 1.2) over 120 min according the USP 28 <711> paddle method (Apparatus 2) at 50 rpm. The artefact dissolved completely.

### Comparative example 22

2.45 g sodium citrate (salt of a tricarboxlic acid) was added to 80.0 g deionized water under simple stirring. 50.0 g Copolymer 1 was suspended in 150 g deionized water and stirred for 10 minutes using a dissolver stirrer at a speed of 550 rpm. Sodium citrate solution was added to the copolymer suspension and stirred for further 24 hours at room temperature and no dispersion was obtained. The degree of neutralization of the anionic polymer was about 3 mol %. After drying a sample at room temperature, a solid, brittle, and opaque inhomogenous artefact was formed. 0.3 g of the artefact was not stable in 0.1 M HCl (pH 1.2) over 120 min according the USP 28 <711> paddle method (Apparatus 2) at 50 rpm. The artefact dissolved completely.

### Comparative example 23

7.38 g sodium citrate was added to 80.0 g deionized water under simple stirring. 50.0 g Copolymer 1 was suspended in 150 g deionized water and stirred for 10 minutes using a dissolver stirrer at a speed of 550 rpm. Sodium citrate solution was added to the copolymer suspension and stirred for further 60 minutes at room temperature until a dispersion was obtained. The degree of neutralization of the anionic polymer was about 9 mol %. After drying a sample at room temperature, a solid, brittle, and opaque film was formed. 0.3 g of the film was not stable in 0.1 M HCl (pH 1.2) over 120 min according the USP 28 <711> paddle method (Apparatus 2) at 50 rpm. The artefact dissolved completely.

### Example 24

7.2 g sodium behenate was added to 80.0 g deionized water and heated to 52° C. under simple stirring. A High viscous aqueous suspension was formed. 50.0 g Copolymer 1 was suspended in 150 g deionized water and stirred for 10 minutes using a dissolver stirrer at a speed of 550 rpm. Sodium behenate suspension was added to the copolymer suspension and stirred for further 60 minutes at room temperature until a viscous dispersion was obtained. The degree of neutralization of the anionic polymer was about 7 mol %. After drying a sample at room temperature, a solid, brittle, and clear film was formed, indicating film forming functionality. 0.3 g of the film was stable in 0.1 M HCl (pH 1.2) over 120 min according the USP 28 <711> paddle method (Apparatus 2) at 50 rpm.

### Example 25

a) 100.0 g of EUDRAGIT® L 30-D55 dispersion was diluted with 16.0 g demineralized water. b) 4.65 g sodium stearate and 1.5 g glycerol mono stearate (GMS 900) was dispersed with 55.7 g demineralized water at 70° C. and added with further 16.0 g demineralized cold water in order to cool the dispersion to 50° C. The dispersion was added to dispersion a) under stirring using a dissolver stirrer at 600 rpm. The degree of neutralization of the anionic polymer was about 9 mol %. After a transient increase of the viscosity for 2 min the dispersion was stirred at 1000 rpm for 30 min in order to obtain a dispersion. When subjecting the disperision to a sieving step using a 0.315 mm sieve 0.06 weight percent retentate is obtained. The dispersion is drying to a flexible, white and glossy film. 0.3 g of the film was stable in 0.1 M HCl (pH 1.2) over 120 min according the USP 28 <711> paddle method (Apparatus 2) at 50 rpm.

### Example 26

a) 3.0 g sodium caprylate was dissolved in 7.0 g demineralized water at room temperature and added to 100.0 g of EUDRAGIT® L 30-D55 dispersion under stirring using a dissolver stirrer at 600 rpm. After a transient increase of the viscosity for 2 min the dispersion was stirred at 1000 rpm for 30 min. b) 1.0 g GMS-SE (self emulsifying glycerol mono stearate) was added to 12.3 g demineralized water and heated to 70° C., vigorously stirred for 2 Min. The degree of neutralization of the anionic polymer was about 10.7 mol %. The suspension b) was cooled down at room temperature and added to dispersion a). The dispersion is drying to a flexible, clear and glossy film. 0.3 g of the film was stable in 0.1 M HCl (pH 1.2) over 120 min according the USP 28 <711> paddle method (Apparatus 2) at 50 rpm.

### Example 27

a) 1.13 g sodium caprylate was dissolved in 15.2 g demineralized water at room temperature and added to 75.0 g of an EUDRAGIT®L 30-D55 dispersion under stirring using a dissolver stirrer at 600 rpm. After a transient increase of the viscosity for 2 min the dispersion was stirred at 1000 rpm for 30 min. b) 0.7 g GMS-SE was added to 16.9 g demineralized water and heated to 70° C., vigorously stirred for 2 Min. The suspension b) was cooled down at room temperature and added to dispersion a). The degree of neutralization of the anionic polymer was about 5 mol %. The dispersion is drying to a flexible, clear and glossy film. 0.3 g of the film was stable in 0.1 M HCl (pH 1.2) over 120 min according the USP 28 <711> paddle method (Apparatus 2) at 50 rpm. When spray coating theophylline pellets using 6% weight gain nearly the same gastric resistant and enteric coating characteristics as in example 6 were obtained.

### Example 28

a) 1.8 g sodium caprylate was dissolved in 6.9 g demineralized water at room temperature and added to 120.0 g of an EUDRAGIT® L 30-D55 dispersion under stirring using a dissolver stirrer at 600 rpm. After a transient increase of the viscosity for 2 min the dispersion was stirred at 1000 rpm for 30 min. b) 1.5 g GMS-SE and 0.26 g Syloid® 244FP were added to 22.4 g demineralized water and heated to 70° C., vigorously stirred for 2 Min. The suspension b) was cooled down at room temperature and added to dispersion a). The degree of neutralization of the anionic polymer was about 5.4 mol %. The dispersion is drying to a flexible, clear and glossy film. 0.3 g of the film was stable in 0.1 M HCl (pH 1.2) over 120 min according the USP 28 <711> paddle method (Apparatus 2) at 50 rpm. When spray coating theophylline pellets using 6 weight gain nearly the same gastric resistant and enteric coating characteristics as in example 6 were obtained.

### Example 29

a) 2.0 g sodium caprylate was dissolved in 4.7 g demineralized water at room temperature and added to 133.3 g of an EUDRAGIT® L 30-D55 dispersion under stirring using a dissolver stirrer at 600 rpm. After a transient increase of the viscosity for 2 min the dispersion was stirred at 1000 rpm for 30 min. b) 1.82 g GMS-SE and 0.2 g Aerosil R972 were added to 38.0 g demineralized water and heated to 70° C., vigorously stirred for 2 Min. The suspension b) was cooled down at room temperature and added to dispersion a). The degree of neutralization of the anionic polymer was about 5.4 mol-%. The dispersion is drying to a flexible, clear and glossy film. 0.3 g of the film was stable in 0.1 M HCl (pH 1.2) over 120 min according the USP 28 <711> paddle method (Apparatus 2) at 50 rpm. When spray coating theophylline pellets using 6 weight gain nearly the same gastric resistant and enteric coating characteristics as in example 6 were obtained.

### Example 30

2.0 g sodium hexanoate was added to 8.0 g demineralized water under simple stirring. A low viscous colloidal solution was formed. 30.0 g Copolymer 1 was suspended in 120 g deionized water and stirred for 30 minutes using a dissolver stirrer at a speed of 550 rpm. Sodium hexanoate suspension was added to the copolymer suspension and stirred for further 240 minutes at room temperature until a dispersion of low viscosity was obtained. The degree of neutralization of the anionic polymer was about 8.6 mol %. After drying a sample at room temperature, a solid, flexible, clear film was formed, indicating film forming functionality. 0.3 g of the film was stable in 0.1 M HCl (pH 1.2) over 120 min according the USP 28 <711> paddle method (Apparatus 2) at 50 rpm.

### Comparative example 31

30.0 g EUDRAGIT® S 100 was added to 100 g demineralized water and stirred using a dissolver stirrer. Subsequently a solution containing 9.2 g sodium caprylate in 57.2 g demineralized water was added. The degree of neutralization of the anionic polymer was about 54.5 mol-%. The dispersion obtained dried at room temperature to form a solid, opaque film was formed. 0.3 g of the film was not stable in 0.1 M HCl (pH 1.2) over 120 min according the USP 28 <711> paddle method (Apparatus 2) at 50 rpm. The film dissolved completely.

### Example 32

7.2 g sodium caprylate was added to 16.8 g demineralized water under simple stirring. A low viscous colloidal solution was formed. 46.4 g hydroxypropyl methyl cellulose phthalate (HP55) was suspended in 277 g deionized water and stirred for 30 minutes using a dissolver stirrer at a speed of 550 rpm. Sodium caprylate suspension was added to the HP55 suspension and homogenized for further 240 minutes using a homogenizer at room temperature until a dispersion of low viscosity was obtained. The degree of neutralization of the anionic polymer was about 50 mol %. After drying a sample at room temperature a clear, flexible film was formed, indicating film forming functionality. 0.3 g of the film was stable in 0.1 M HCl (pH 1.2) over 120 min according the USP 28 <711> paddle method (Apparatus 2) at 50 rpm.

### Comparative example 33

0.6 g sodium caprylate was added to 8.0 g demineralized water under simple stirring. A low viscous colloidal solution was formed. 30.0 g Copolymer 1 was suspended in 90.9 g deionized water and stirred for 30 minutes using a dissolver stirrer at a speed of 550 rpm. Sodium caprylate suspension was added to the copolymer suspension and stirred for further 240 minutes at room temperature and no dispersion was obtained. The degree of neutralization of the anionic polymer was about 2 mol %.

### Example 34

2.8 g sodium-2-hydroxy-octanoate was added to 8.0 g demineralized water under simple stirring. A low viscous colloidal solution was formed. 30.0 g Copolymer 1 was suspended in 120 g deionized water and stirred for 30 minutes using a dissolver stirrer at a speed of 550 rpm. Sodium-2-hydroxy-octanoate suspension was added to the copolymer suspension and stirred for further 240 minutes at room temperature until a dispersion of low viscosity was obtained. The degree of neutralization of the anionic polymer was about 9 mol %. After drying a sample at room temperature, a solid, flexible, clear film was formed, indicating film forming functionality. 0.3 g of the film was stable in 0.1 M HCl (pH 1.2) over 120 min according the USP 28 <711> paddle method (Apparatus 2) at 50 rpm.

### Comparative example 35

2.2 g caprylic acid was added to 8.0 g demineralized water under simple stirring. A low viscous emulsion was formed. 30.0 g Copolymer 1 was suspended in 120 g deionized water and stirred for 30 minutes using a dissolver stirrer at a speed of 550 rpm. Caprylic acid emulsion was added to the copolymer suspension and stirred for further 240 minutes at room temperature and no dispersion was obtained. The amount of caprylic acid corresponds to a degree of neutralization of the anionic polymer was about 9 mol %.

### Comparative Example 36

4.3 g stearic acid was added to 8.0 g demineralized water under simple stirring. A high viscous suspension was formed. 30.0 g Copolymer 1 was suspended in 120 g deionized water and stirred for 30 minutes using a dissolver stirrer at a speed of 550 rpm. Stearic acid suspension was added to the copolymer suspension and stirred for further 240 minutes at room temperature and no dispersion was obtained. The amount of stearic acid corresponds to a degree of neutralization of the anionic polymer was about 9 mol %.

### Example 37

3.4 g sodium caprylate and 12.0 g sodium stearate were dissolved in 480 g demineralized water at room temperature and added to 120.0 g of EUDRAGIT® L 100-55 dispersion under stirring using a dissolver stirrer at 600 rpm. After a transient increase of the viscosity for 2 min the dispersion was stirred at 1000 rpm for 30 min. The degree of neutralization of the anionic polymer was about 9 mol %. The dispersion is drying to a flexible, clear and glossy film. 0.3 g of the film was stable in 0.1 M HCl (pH 1.2) over 120 min according the USP 28 <711> paddle method (Apparatus 2) at 50 rpm.

1. A composition, comprising:
(a) an anionic polymeric material, and
(b) a salt of a saturated monocarboxylic acid, comprising from 6 to 22 carbon atoms,
wherein an amount of all salt (b) is from 3 to 50 mol percent of an amount of anionic groups in the polymeric material.

2. The composition of claim 1, further comprising:
(c) a pharmaceutically, nutraceutically, or cosmetically acceptable excipient different from the anionic polymeric material (a) and different from the salt (b),
wherein the excipient (c) is at least one excipient selected from the group consisting an antioxidant, a brightener, a flavoring agent, a flow aid, a fragrance, a glidant, a penetration-promoting agent, a pigment, a plasticizer, a polymer, a pore-forming agent, and stabilizers.

3. The composition of claim 1, as a powder.

4. The composition of claim 1, as an aqueous dispersion, suspension, or solution.

5. The composition of claim 1, wherein the polymeric material (a) is an anionic (meth)acrylate copolymer comprising a free-radical polymerized unit,
wherein the free-radical polymerized unit comprises from 25 to 95% by weight of a C₁- to C₄-alkyl ester of acrylic or of methacrylic acid and from 5 to 75% by weight of a (meth)acrylate monomer comprising an anionic group.

6. The composition of claim 1,
wherein the anionic polymeric material (a) is partially neutralized by an alkaline agent,
the alkaline agent is not a salt (b) of the saturated monocarboxylic acid, and
the alkaline agent is selected from the group consisting of an alkali metal salt and an ammonium salt.

7. The composition of claim 1, wherein the salt (b) is selected from the group consisting of caproic acid, ornathic acid, caprylic acid, pelargonic acid, caprinic acid, lauric acid, myristic acid, palmitic acid, margaric acid, stearic acid, arachidic acid, behenic acid, and mixtures thereof.

8. The composition of claim 7, wherein the salt (b) is sodium caprylate or sodium stearate.

9. A matrix formulation, comprising:
the composition of claim 1;
a pharmaceutically, nutraceutically, or cosmetically active ingredient; and
optionally, a further excipient.

10. A process for preparing an aqueous coating dispersion or solution comprising the composition of claim 1, wherein the process comprises:
neutralizing an anionic group of the anionic polymeric material (a) with the salt (b) to a degree of 3 to 50 mol percent, and
combining the anionic polymeric material (a), and the salt (b), and water, and
mixing the anionic polymeric material (a), the salt (b), and water to obtain the aqueous coating dispersion or solution.

11. The process of claim 10, further comprising:
adding to the dispersion at least one pharmaceutically, nutraceutically or cosmetically acceptable additive selected from the group consisting of a pigment, a release agent, a plasticizer, and an emulsifier.

12. The process of claim 10, further comprising:
reacting, in situ, (1) a saturated monocarboxylic acid comprising from 6 to 22 carbon atoms with (2) an alkali metal hydroxide or an ammonium hydroxide, to obtain the salt (b).

13. The process of claim 12, comprising:
mixing the anionic polymeric material (a) as an organic solution with the salt (b) by wet granulation.

14. A gastric resistant dosage form, comprising:
a core comprising a pharmaceutical, nutraceutical, or cosmetically active ingredient, and
a coating layer on at least a part of said core comprising the composition of claim 1.

15. A coating or binding agent comprising the composition of claim 1.

16. The composition of claim 1, wherein the salt (b) comprises an alkali metal cation or ammonium.

17. The composition of claim 1, wherein the salt (b) comprises a salt of caprylic acid.

18. The composition of claim 5, wherein a glass transition temperature of the anionic (meth)acrylate copolymer is from 55 to 70° C.

19. The dosage form of claim 14, further comprising:
a second coating, on top of the coating layer.

20. The dosage form of claim 14, further comprising:
a second coating, beneath the coating layer.
